(19) United States
(12) Patent Application Publication
(10) Pub. No.: US 2020/0102074 A1
(43) Pub. Date: Apr. 2, 2020

(54) A DEVICE, METHOD AND SYSTEM FOR AN UNMANNED AERIAL VEHICLE HEALTH CARE COMPANION

(71) Applicant: SAINT ELIZABETH HEALTH CARE, Markham (CA)

(72) Inventors: Allyson KINSLEY, Toronto (CA); Roy FRENCH, Toronto (CA); Jeff CORSIGLIA, Sooke (CA); Grant MCKEE, Red Deer (CA); Paolo KORRE, Toronto (CA)

(73) Assignee: SAINT ELIZABETH HEALTH CARE, Markham, ON (CA)

(21) Appl. No.: 16/497,877

(22) PCT Filed: Apr. 26, 2018
(86) PCT No.: PCT/CA2018/050491

§ 371 Date: Sep. 26, 2019

Related U.S. Application Data
(60) Provisional application No. 62/490,122, filed on Apr. 26, 2017.

(51) Int. Cl. B64C 39/02; G10L 15/22; G06F 3/16; G08G 5/00

(52) U.S. Cl. CPC B64C 39/024; G10L 15/22; G06F 3/167; B64C 2201/141; B64C 2201/146; G10L 2015/223; B64C 2201/127; G08G 5/0069

(57) ABSTRACT

The present invention is a companion unit and companion system. The companion unit comprises an UAV that further comprises one or more of the following elements: propulsion units, circuit boards, navigation sensors, cameras, speakers, microphones, chassis, processors, and batteries. The companion system comprises one or more bases wirelessly connected to the companion unit and in possibly to one or more computer devices and/or to the Internet. The elements thereby facilitate one or more of the following Internet connectivity, wireless local area network (WiFi) connectivity, computer device connectivity (e.g. connectivity to a cell phone, a smart phone, a laptop, a tablet, a smart television, a WiFi enabled appliance, or any other computer device), base connectivity, and cloud storage connectivity. The companion system and/or the companion unit is connected to an artificial intelligence module. The companion unit and system are programmed to function as a companion to a human user.

This application claims the benefit of U.S. Provisional Patent Application Ser. No. 62/490,122 filed Apr. 26, 2017.

### FIELD OF INVENTION

This invention relates in general to the field of health care companions and more particularly to unmanned aerial vehicle (UAV) artificial intelligence health care companions and systems.

### BACKGROUND OF THE INVENTION

The field of technology and prior art inventions focused upon heath information, and health information tracking in particular, have been developing over many years. Such technologies can integrate biometric sensors that capture a human's biometric information, such as heart rate. An example of this type of technology is a wearable device that incorporates sensors, such as biometric sensors, that operate to capture information, such as heart rate information. For example, U.S. Patent Application Publication No. 20170084133 filed by Apple Inc. on Aug. 29, 2016 discloses a wearable device that comprises a band and a housing that incorporates a sensor, such as a biometric sensor.

Health field technologies have also developed to incorporate some artificial intelligence. For example, U.S. Patent Application Publication No. 2017008419 filed by Mark E. Nusbaum and Vincent Pera on Nov. 26, 2016, discloses the use of artificial intelligence-based software that automatically responds to medical queries and sends warning messages if maximum or minimum thresholds are exceeded or are not met. Such responses may be triggered by, for example, a user consuming too much sugar, too many calories, not completing enough exercise, etc.

Technology has further been developing relating to unmanned aerial vehicles (UAVs) that assist with human activities. For example, U.S. Pat. No. 9,471,059 granted to Amazon Technologies Inc. on Oct. 18, 2016, discloses UAVs programmed to provide assistance to a user. Specifically, the assistance that the UAVs provide relates to acting as eyes and/or ears for the user by providing viewing and listening features once travelling to areas that are not accessible by the user. The UAV records information from a perspective that is different from the user's perspective, and can thereby assist with scouting dangerous situations, locating items, and checking the status of certain activities (i.e., the boiling of water, and the running of a dryer).

As further examples of such UAV related prior art: Amazon™ has developed UAVs that hover and can communicate through sound with a user; Google™ has developed remote telepresence UAVs; Spinmaster Toys™ have developed UAVs for indoor use; Fleye™ have developed hovering telepresence UAVs; and 3D Robotics Iris+™ has developed a follow-me function for UAVs.

As another example of aerial prior art, U.S. Pat. No. 9,409,645 granted to Google, Inc. on Aug. 9, 2016, discloses a mobile telepresence system. The system includes a frame, a propulsion system coupled to the frame, a screen movably coupled to the frame, and an image output device coupled to the frame. The propulsion system operates to propel the frame through a designated space, and the image output device may project an image onto the screen in response to an external command.

### SUMMARY OF THE INVENTION

In one aspect, the present disclosure relates to a companion system operable to interact with a user, comprising: a companion unit that is an unmanned aerial vehicle incorporating one or more navigational sensors, one or more cameras, one or more speakers and one or more microphones; one or more bases wirelessly connected to the companion unit and to the Internet; and an artificial intelligence module connected to the companion system operable to process commands and provide instructions to the companion system to be performed by the companion unit.

In another aspect, the present disclosure relates to a companion system that also comprises one or more navigational sensors that include one or more proximity sensors operable to recognize obstacles in the flight path of the companion unit.

In another aspect, the present disclosure relates to a companion system operable to interact with a user, comprising: a companion unit that is an unmanned aerial vehicle incorporating one or more navigational sensors, one or more cameras, one or more speakers and one or more microphones; one or more bases wirelessly connected to the companion unit; and an artificial intelligence module (AI Module) connected to the companion system operable to process commands and provide instructions to the companion system to be performed by the companion unit.

The companion system further comprising the one or more navigational sensors that include one or more proximity sensors operable to recognize obstacles in the flight path of the companion unit.

The companion system further comprising one or more of the following being connectable via an Internet connection: the companion unit; and at least one of the one or more bases.

The companion system further comprising one or more mobile devices remotely located from the companion unit being connectable to one or more of the following via the Internet: the companion unit; and at least one of the one or more bases, said one or more mobile devices being operable by one or more remote users to communicate with the companion unit and to the user via the companion unit.

The companion system further comprising the one or more remote users being one or more of the following: a care giver; a health care provider; a family member; or a friend.

The companion system further comprising one or more of the following being connectable to one or more computer devices: the companion unit; and at least one of the one or more bases.

The companion system further comprising the one or more computer devices being one or more of the following: a cell phone, a smart phone, a laptop, a tablet, a smart television, and a WiFi enabled appliance.

The companion system further comprising a flight controller connected to the companion unit, whereby the in-flight speed and direction of a companion unit may be controlled.

The companion system further comprising the one or more sensors connected to a location and mapping module operable to generate a map local to the location of the companion unit.

The companion system further comprising a flight plan guidance unit operable to receive information from the AI Module and the map, and to process such information and the map to generate a flight path for the companion unit.

The companion system further comprising a voice assistant operable to receive voice commands provided via the microphone of the companion unit, and to process said voice commands to cause the companion unit to respond by flying a mission or generating a voice response provided to the user via the speakers of the companion unit.

In yet another aspect, the present disclosure relates to a method of one or more operating users operating a companion unit, comprising the following steps of: one of the one or more operating users providing a command to the companion unit; the companion unit transmitting the command to an artificial intelligence module (AI Module) and the AI Module processing the command to generate a response that is transmitted to the companion unit; and the companion unit operating in accordance with the response.

The method comprising the further step of one of the one or more operating users, being a local user in proximity to the companion unit, providing the command as a voice command to a microphone of the companion unit.

The method of comprising the further steps of: one of the one or more operating users, being a remote user remotely located from the companion unit, providing the command as a voice command to a microphone in a computing device of said remote user; the computing device transmitting the voice command to one of the following: a microphone of the companion unit, and the microphone transmitting the voice command to the AI Module; or the AI Module; and the AI Module processing the voice command as the command to generate the response.

The method comprising the further steps of: one of the one or more operating users, being a remote user remotely located from the companion unit, providing the command as an input command by inputting the input command to a computing device of said remote user; the computing device transmitting the input command into the AI Module; and the AI Module processing the input command as the command to generate the response.

The method comprising the further step of the AI Module transmitting information to a flight controller, and said flight controller controlling a motor of the companion unit to control the flight of the companion unit that is the response to the command.

The method comprising the further steps of: one or more sensors transmitting sensor information to the flight controller; and the flight controller processing such sensor information to control the flight of the companion unit.

The method comprising the further steps of: one or more sensors transmitting sensor information to a location and mapping unit and the location and mapping unit processing the sensor information to generate a local map of an area proximate to the companion unit; the location and mapping unit transmitting the map to a flight guidance unit; the AI Module transmitting command information to the flight plan guidance unit; the flight plan guidance unit processing the command information and map to generate a flight path within the map for the flight of the companion unit that is the response to the command; and the companion unit flying along the flight path to execute the response.

The method comprising the further steps of the AI Module transmitting information to one or more databases; and the AI Module accessing information from the one or more databases, said information being utilized by the AI Module in processing the command and generating the response.

The method comprising the further steps of: one or more cameras incorporated in the companion unit generating video of an area proximate to the companion unit and transferring said video to one or more computer devices for viewing by the one or more operating users; the one or more operating users communicating with each other via microphones and speakers incorporated in the companion unit; and the companion unit engaging in conversation with at least one of the one or more operating users.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The invention will be better understood and objects of the invention will become apparent when consideration is given to the following detailed description thereof. Such description makes reference to the annexed drawings wherein:

FIG. 1 is a top view of the companion unit, in accordance with an embodiment of the present invention.

FIG. 2 is a cross-sectional view of the companion unit, in accordance with an embodiment of the present invention.

FIG. 3 is a top view of the companion unit indicating function of sensors therein, in accordance with an embodiment of the present invention.

FIG. 4 is a side perspective view of the companion unit in-flight above a surface indicating function of an altitude sensor (altimeter) therein, in accordance with an embodiment of the present invention.

FIG. 5 is a systems diagram of a configuration of elements of the companion system within multiple rooms, in accordance with an embodiment of the present invention.

FIG. 6 is a view of the companion unit within a room comprising multiple obstacles, in accordance with an embodiment of the present invention.

FIG. 7 is a systems diagram of a configuration of elements of the companion system incorporating cloud storage, in accordance with an embodiment of the present invention.

FIG. 8 is a systems diagram of a configuration of elements of the companion system as utilized by a local user and a remote user, in accordance with an embodiment of the present invention.

FIG. 9 is a systems diagram of a configuration of elements of the companion system operable to produce video output, in accordance with an embodiment of the present invention.

FIG. 10 is a systems drawings of a flight controller of an embodiment of the present invention.

FIG. 11 is a systems drawings of a function system of an embodiments of the present invention.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION OF THE PREFERRED EMBODIMENT

The present invention is a companion unit and companion system. The companion unit comprises an UAV that further comprises one or more of the following elements: one or more propulsion units, one or more circuit boards, one or more navigation sensors, one or more cameras, one or more speakers, one or more microphones, one or more chassis, one or more processors, and one or more batteries. The companion system comprises one or more bases wirelessly connected to the companion unit and possibly to one or more computer devices. The companion unit, the one or more bases and/or the one or more computer devices may be Internet enabled and may be connected to the Internet.

The terms “information” and “data” as used herein to mean all forms and types of information and data. All references herein to any transmission, transfer or other communication of information or data between elements of the present invention, means transmission of data that may be via a wired or wireless connection between the elements of the present invention. The transmission of data or information between elements of the present invention may further be direct between the elements, or via the Internet or cloud services.

The elements of the companion unit and system are operable to facilitate one or more of the following: Internet connectivity, wireless local area network (WiFi) connectivity, computer device connectivity (e.g. connectivity to a cell phone, a laptop, a tablet, a smart television, a WiFi enabled appliance, or any other computer device), base connectivity, and cloud storage connectivity.

The companion system and/or the companion unit is connected to an artificial intelligence module. The companion unit and system may have video capture and output capabilities, as well as data capture, processing and transfer capabilities.

The companion unit and system are programmed to function as a companion to a human user. The companion unit and system operate to interact with the user through data capture by the unit and output provided to the user.

In functioning as a companion, the present invention is operable to stave off loneliness and isolation in a user (who is the person to whom the companion unit and system function as a companion). Loneliness and isolation are recognized as significant problems in aging populations that can affect both the mental and physical health of an individual.

The present invention can provide companionship, conversation, entertainment, security, and health monitoring. Through being present with the user and interacting with the user the companion unit and system may thereby ameliorate, alleviate or avoid loneliness and isolation of the user. In this manner, the present invention can act as a therapeutic device that helps a user to avoid detriments to his or her mental and/or physical health that can be caused by loneliness and/or isolation.

The companion unit and system are WiFi and/or Internet enabled. The memory of the companion unit and system can be incorporated in one or more of: cloud storage; hard drive storage; and/or sever storage. The storage can be integrated in one or more of the following: the companion unit; the bases of the companion system; one or more computer devices (e.g., cell phone, smart phone, laptop, computer, tablet, a smart television, a WiFi enabled appliance, or other computer device) connected to the companion unit and/or companion system; and/or a server.

In embodiments of the present invention can communicate with and integrate with the internet of things (e.g., internet enabled devices located proximate to the companion unit and system). The companion unit and system can therefore communicate with any internet enabled device, including internet of things medical devices, internet of things home appliance devices, internet of things televisions or other entertainment devices, internet of things vehicles, or any other internet of things connected device. Processing requirements of the companion unit and system can be achieved by one or more of processors integrated with: the companion unit; the companion system; one or more computer devices connected to the companion unit and system; and/or a remote system. The processors may transfer information to and from the companion unit and system elements via the Internet, WiFi connectivity or any other connection operable to achieve such transmission.

Embodiments of the present invention may incorporate a handheld controller that the user may utilize to operate certain functions of the companion unit and system. In such an embodiment, the companion unit is operable to transmit audio and/or video to the user's hand held controller. The companion unit may further be operable to transmit audio and/or video to one or more computer devices accessible by the user, or accessible by a care giver of the user, health care provider of the user, or other remote user. When sending audio and/or video to a remote care giver, health care provider, or other remote user the companion unit and system may be operable to send such audio and/or video to a remote computer device via WiFi, cellphone or Internet connectivity.

The companion system may incorporate an artificial intelligence personality interface module. The companion unit and system may be operable to provide a user with a conversationally proficient voice responsive flying home companion. The companion unit and system may learn the preferences of the user, and thereby provide conversation, entertainment and other interaction with the user that is tailored to the user's personality and preferences.

The companion unit and system can perform a variety of activities. For example, the companion unit can move in proximity to a user, such as by following the user. The companion unit can also precede the user and provide a video preview of the route that the user is advancing towards. The companion unit can also be tasked to fly along a flight path to a remote location. The flight may be for the purpose of a mission. For example, once at the location the companion unit may monitor the location through video surveillance, or perform a task, for example, such as determining the status of a window (i.e., closed or open), or a stove (i.e., turned on or turned off).

Such tasking of the companion unit can occur due to voice commands directed to the companion unit and system, or control of the unit by a person or by the system. As an example, a voice command may be “Go see if I left the stove on” to cause the companion unit and system to check the status of the stove. Another voice command may be “follow me” to cause the unit to move in proximity to, and behind, the user. Proximity of the companion unit to a user may be determined through use of sensors, as described herein.

The user can further ask the companion unit and system to provide web based or streaming entertainment, to call a friend, to ask a person (e.g., a caregiver, family member, health provider, etc.) or an organization (e.g., a hospital, an emergency call centre, a home security call centre, etc.) for help on behalf of the user, to play audio books, podcasts, movies, or music.

The user can further ask the companion unit and system to control WiFi enabled home appliances, or to take control of a smart phone and access its features. In some embodiments of the present invention this may be achieved through a connection with the internet of things.

Moreover, the companion unit can initiate conversations with the user or other humans, ask and answer questions, propose entertainment media to a user, and play games with the user. The companion unit may further be operable to phone a family member, caregiver, health care professional, or other party at the request of the user.

The companion unit and system can remind the user of calendar events, routine event (e.g., when to take medications, etc.), or when to perform other tasks or activities.

The companion unit and system can also provide family members, caregivers and/or health care professionals (remote users) with a tool for monitoring users, such as through video surveillance, audio surveillance, confirmation that the user has undertaken certain tasks (e.g., the user has taken medications on time, the user has bathed, the user has eaten meals, the user has taken a walk, etc.).

The companion unit may have the appearance of a character, such as a bird or other animal, or some other character that may be human or non-human.

The companion unit is mobile, in that it can fly and hover. It can also rest against surfaces, such as a chair, a couch, a countertop, a table, or upon other surfaces able to bear the size and weight of the companion unit. The companion unit can therefore be positioned close to a user when the companion unit is in a static posiiton. It can follow a user as the user moves within a room, a building, or outside. It can also be sent to certain locations by the user, such as through rooms in a building, to check if a window is closed, etc. The companion unit can be programmed to remain in relative proximity to a specific user.

The companion unit may incorporate one or more sensors that are navigational sensors. The navigational sensors may function such that the companion unit can sense its proximity to walls, floors, furniture, appliances, countertops, and other impediments or obstacles to the travel by flight of the companion unit. By sensing such obstacles the companion unit can adjust the trajectory of its travel path, and thereby fly within spaces without contacting any such obstacles. The sensors may further permit the companion unit either individually, or with assistance of the companion system, to fly within a space without a human operator directing its flight. If a companion unit is flying proximate to a person, some of its actions, such as hovering, landing in a position, or flying, may be triggered by movements of the user, so as to cause the companion unit to maintain a proximity between the user and the companion unit.

The companion unit may be operable to use navigational sensors to map its environment, avoid obstacles, identify objects and people, and hold altitude, among other activities. Cloud storage may be utilized to enable complex mapping of the environment of the companion unit and the locations of any obstacle(s) therein.

In embodiments of the present invention, beacons may be located in an environment where the companion unit is operated. For example, the beacons may be Bluetooth™ beacons, or other types of beacons. The beacons may incorporate small transmitters, such as radio transmitters, that send out signals in a specific radius within the environment. As an example, the radius may be of 10-30 meters within an interior space, or some other radius. Beacons may be operable to determine the position of the companion unit. For example, a beacon may determine the position of the companion unit with an accuracy of up to 1 meter, or to some other level of accuracy. Beacons can be built to standards that are very energy efficient. The companion unit can sense the beacons to detect the current floor or other surfaces below the companion unit in the environment.

Bluetooth beacons may be preferable because they normally do not affect other radio networks, and interference can thereby be avoided. For example, such beacons do not interfere with medical devices. It is possible for beacons to experience interference with WiFi signals, but this problem can be addressed through the selection of the channels for which the WiFi signal is configured, as will be recognized by a skilled reader. The beacons may utilize the available channels that WiFi is set not to use, and the beacons may utilize such channels to capacity in a uniform manner, such as may involve frequency hopping.

The beacons function in that the intersection of the beacon with the companion unit can be processed to indicate the location of the companion unit in relation to the beacons. The location of the beacons within an environment can be mapped. The information about the intersection of the beacons with the companion unit, can be processed to identify the location of the companion unit with an environment. The location can then be depicted on the map. The proximity of the companion unit to any obstacle, can also be determined, such as in accordance with mapped obstacles, and the companion unit can navigate within an environment to avoid obstacles based upon such information.

The companion unit may be operable to provide information to the user, including information that is in the form of speech. The companion unit, through its operability with the companion system, may incorporate a learnable/teachable artificial intelligence element, whereby it can build a personality based upon multiple interactions with the user. The companion may thereby initially convey specific information to the user, as it is prompted to do by the companion system, but over time it may learn to converse with the user, such that the conversation operates beyond merely transmitting or requesting specific information. In this manner the companion unit and system may provide company and mental stimulation to a user, as well as providing information to the user. Thus, the interaction of the user and the companion unit will not be limited to perfunctory exchanges, such as reminders to undertake tasks (e.g., reminders to take medications, etc.), but will include conversation that is organic and is not scripted.

The companion unit may capture information, such as sounds within a space, including speech or other noises made by a user. For example, the companion unit may capture questions asked by the user, such as questions about the user's medications. The sounds may be captured via microphones incorporated in the companion unit.

The companion unit may further capture images or video footage within a space, including images or video showing the user. For example, the companion unit may capture images or video of the user taking medications. The companion unit may thereby capture confirmation that medications were ingested by the user, as well as specific details such as the date and time when the medications were ingested by the user. Another example is that the companion unit can capture images or video of a window. Upon viewing such images or video the user can confirm if a window is open or closed. This can assist a user in that the user does not have to walk to the window to check if it is open or closed. These are but some examples of images or video that may be captured by the companion unit, and how such images or video may be processed and interpreted by the companion unit and system to generate information relating to the user.

The companion unit may capture, record and store such information and it may relay such information to the companion system at a later point in time. For example, the information may be relayed upon the companion unit connecting with the base station for recharging, or when the companion unit is proximate to the base station. Alternatively, the companion unit may immediately relay such information to the companion system, through WiFi transmission, or some other form of transmission, if a base unit or a computer device linked to the companion system is sufficiently proximate to receive such a transmission. The companion system or the computer device to which such information is transmitted may process the information as described herein.

Such information may be directed to other parties by the companion system. For example, a user's question about medication may be directed by the companion system to a doctor or a nurse, and the response to the query may be provided directly to the user, or may be provided to the companion system whereby the companion unit will provide the response to the user. As another example, the companion unit may transfer video showing the user taking medications to a designated family member, or other caregiver of the user who is located remotely from the user. In this manner the remotely located family member or caregiver can monitor the user's activities from afar, to ensure the user undertakes activities required to maintain and support the user's health (e.g., medications, health regimes such as exercise and bathing, or other activities).

The companion unit and system offer several advantages over the prior art, including that the companion unit's ability to fly allows it to capture information relating to a user from several angles that are not achievable by wearable technologies. For example, the companion unit can capture images or video footage from behind the user, and therefore may capture images that can be processed by the companion system to help determine if a user is limping or showing other signs of injury, or shows evidence that the user is healing from an injury. This information can be of great assistance to remote users in monitoring the health of a user.

The companion unit and system further offer the benefit that they can develop a personality that can undertake conversation that is beyond merely directing specific scripted questions, such as health questions, to a user. This is achieved through the integration of learnable/teachable artificial intelligence in the companion unit and system.

The companion unit and system thereby creates an enjoyable user experience. The user is not merely interacting with a device programmed to obtain and transmit specific information. The user is interacting with a device that has and is constantly building a personality. Such exchanges may therefore include exchanges between the user and the companion unit that are neither scripted nor specifically required to transmit or receive health information. The user can converse with the companion unit, and thereby have the experience of constantly having company nearby, even if there is no other human in the vicinity of the user.

This aspect of the present invention further increases the effectiveness of the companion unit and system in providing companionship to the user that can thwart loneliness and isolation of the user. As discussed herein, by providing companionship to a user, the user can avoid mental and physical health detriments that can be caused by loneliness and isolation. The companion unit can further be utilized to connect the user with other people who are located remotely from the user, and thereby further decrease the loneliness and isolation of the user.

Moreover, a further benefit of the companion unit and the system is that due to the varied information that it can collect and transmit (i.e., aerial images and video, health information, verification of the ingestion of medications, relaxed conversational exchanges, etc.), it can achieve processing of information and results therefrom that are not achievable without such information. Healthcare professionals, family members, remote caregivers, and other authorized persons (remote users), can be linked into the companion system, and can thereby benefit from such information. For example, a remote caregiver can be provided with confirmation that all prescribed medications are or are not ingested by the user, and that the user is enjoying a day with limited symptoms related to an illness, or is suffering one or more symptoms. The user can thereby be monitored without physical interference from a caregiver, and can maintain more autonomy and dignity than may otherwise be possible. Moreover, as the interactions with the companion unit occur throughout a day, the stress that the user may otherwise feel in relation to a caregiver's visit is not experienced by the user because the caregiver can monitor the user without having to visit the user as regularly. Thus, the companion unit and system can improve the quality of life for the user and the caregiver.

The companion unit as described herein, may replace human interactions in a common environment in some instances by allowing users to interact with persons who are remotely located from the user. However, the companion unit can also be used to supplement human interactions with the user. In a supplemental role, the companion unit is operable to facilitate interactions between a user and another person remotely located from the user. This may be a measure that is implemented to ensure regular interactions between the user and certain people who the user would normally interact with in the user's environment (the person and the user are physically in the same environment), to ensure this interaction is not interrupted when the person is located remotely from the user. When the person cannot be physically located in the same environment as the user the companion unit would act as a supplement to physically proximate interactions.

The companion unit and system can also have specific application for health care providers located remotely from patients. For example, the companion unit and system can be controlled by a healthcare provider to collect images and video of the user from various angles as required to determine symptoms and mobility related information pertaining to the user. The healthcare provider can also speak with the user directly to receive and transmit real-time or virtually real-time information. In some embodiments of the present invention biometric information may be transmitted by the companion system, such as heart rate information, blood analysis information, blood pressure information, activity information, or other information if the sensors, devices, apps or other collectors of this information are connected to the companion system. For example, diabetic blood glucose meters, wearable technologies that track activity and/or heart rate, blood pressure devices, and other sensors, devices, apps or other collectors of biometric and health information may be connected to the companion system. The healthcare user may further receive information captured by the companion unit and system, such as whether the user took medication, whether the user engaged in exercise, and other health activity related information.

As discussed herein, if the sensors, devices, apps or other collectors of biometric information are Internet enabled the interaction of the companion unit and system with such devices may be through the internet of things.

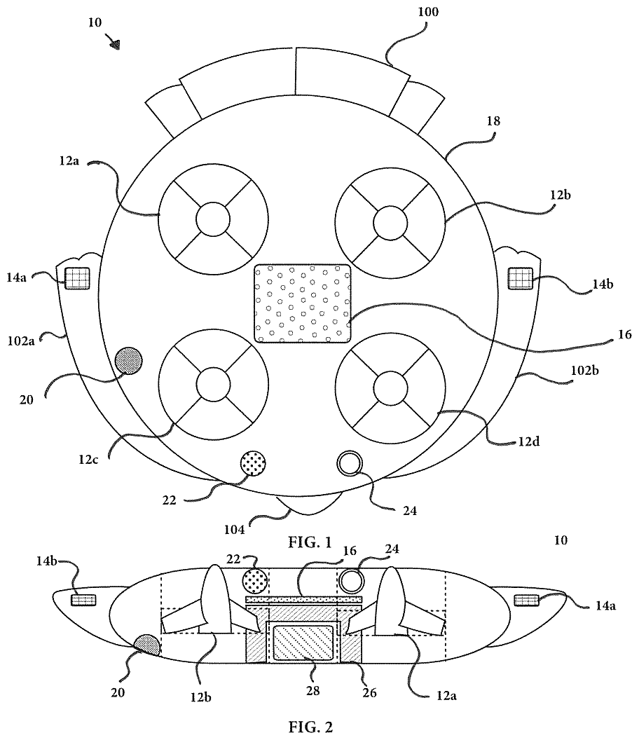

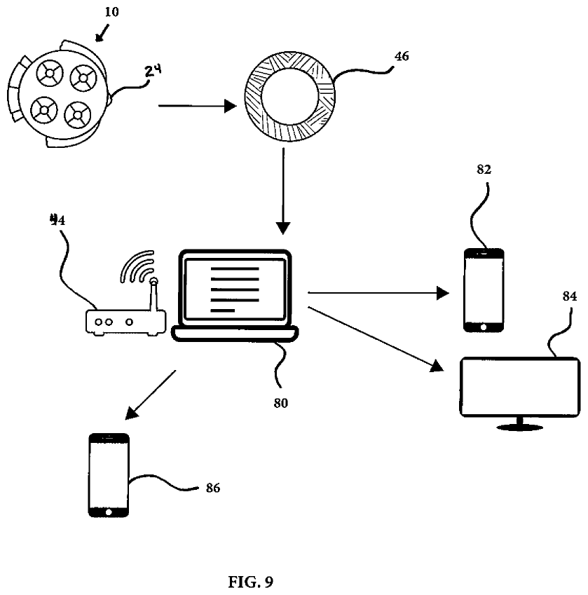

Descriptions of specific embodiments of the present invention are provided herein in reference to FIGS. 1-9. A skilled reader will recognize that other embodiments of the present invention are also possible. The description herein provides information relating to multiple embodiments of the present invention.

As shown in FIG. 1, the companion unit 10 may be shaped like a character, for example, such as a bird, having a beak 104, wings 102a, 102b and a tail 100. A skilled reader will recognize that the companion unit can be shaped as a variety of characters, or the companion unit may be a UAV having the other elements described herein attached to it directly without any character aspects being incorporated within or upon the UAV.

A companion unit that is shaped like a character, may be shaped as, for example, a human figure, an animal, or some other character. The character may be sculpted from a layer 18 attached to the UAV wherein the additional elements in the companion unit are integrated or otherwise attached. The layer may be of foam, or any other rigid or semi-rigid material, such as carbon fibre, fiberglass, aerogel, or expanded bead foams, such as polyolefin, polystyrene or polyethylene.

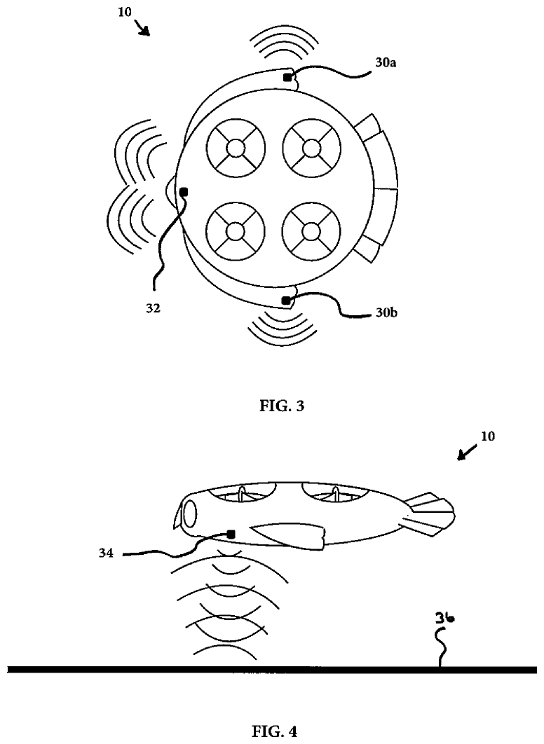

The sculpting of the layer will accommodate the airflow requirements of the propulsion units, and any components thereof, including any fans or shrouded propellers. For example, the propulsion units may be positioned within cylindrical holes in the companion unit, as shown in FIGS. 1 and 2.

The integration of fans or propellers within the companion unit may be configured to be protected for safety and efficiency, as well as for crashworthiness. For example, the sculpted body may be made of foam that provides impact protection and encloses the propulsion units making them safer than exposed blades. As further examples, in some embodiments of the present invention a screen may be positioned over the air intake to filter foreign objects and thereby prevent such foreign objects from entering the interior of the companion unit. In some embodiments of the present invention, a current draw shut-off may be incorporated in the companion unit whereby if the unit collides with any surface and deformation of the unit occurs, the shut-off may occur automatically to prevent further damage to the unit. Furthermore, in some embodiments of the present invention a duct may be configured to control compression and expansion of airflow to increase the efficiency of the companion unit.

The sculpted layer of the companion unit may further be formed to accommodate the aerodynamic requirements for the flight, hovering and maneuverability functions of the companion unit. The sculpted layer may further be configured to not interfere with the stability of the companion unit while in-flight, hovering or at rest.

The companion unit may incorporate one or more propulsion units 12a, 12b, 12c, 12d operable to assist the companion unit to achieve and maintain-flight. The unit may further incorporate one or more navigation sensors 14a, 14b, operable to sense surfaces or objects proximate to the sensors, such as walls, doors, ceilings, floors, stairs, furniture, trees, plants, or other surfaces or objects. The unit may further incorporate a circuit board 16 operable to receive and transmit data to and from the companion system and to collect and process data, as described herein. In some embodiments of the present invention the circuit board may integrate, or otherwise be connected to, at least one processor and at least one data storage unit. Software may be stored in the storage unit and be operable by the processor. Such software may operate functions of the companion unit described herein, and the WiFi or other connectivity of the companion unit may facilitate updates and upgrades to the software being delivered to the companion unit.

In some embodiments of the present invention specific software may be loaded into the storage unit of the companion unit that operates the functions of the companion unit.

Other software operable to process data and information collected by the companion unit may be stored in and operable by a base unit, a computer device, or a remote server.

In still other embodiments of the present invention, the software required to control the companion unit and system may be stored and operated from a remote server.

The unit may further incorporate one or more speakers 20 operable to emit sounds to the user, such as music, speech, or other sounds. The unit may further incorporate one or more microphones 22 operable to amplify, and cause to be recorded, sounds directed to the companion unit. Such sounds may include sounds of a human speaking or making other noises that occur in the environment where the companion unit is located.

The companion unit may further incorporate one or more cameras 24 that can be used to capture still images and/or video. In embodiments of the present invention, one or more of the one or more cameras of the present invention may be connected to software that is operable to perform facial recognition. In this manner, a camera of the present invention may be operable to capture an image of a person and to identify the person in the image. The artificial intelligence module of the present invention may further be operable with the facial recognition module, whereby the companion unit and system can learn to identify people, to recognize facial expressions (such as expressions of happiness, distaste, fear, distress, etc.) of the user and/or other people, as well as other information that can be gathered from a person's face. This information can increase the scope of the interactions of the companion unit with the user and other people in that the interaction can be generated for specific people and can be generated in response to a person's expression, and possibly the person's mood if the expression indicates the person's mood.

As another example of use of facial recognition operable integrated in the companion unit and system, if a person comes to visit the user, the camera may capture an image of that person and may be able to use facial recognition operations to identify that person. The unit and system may thereby be operable to track the persons who visit the user, and may further be operable to interact with the visitor thereby learning the visitor's preferences such that the interaction with the visitor may be tailored in a similar manner as is described herein whereby the unit's interaction with the user may be tailored. The facial recognition operability may further be utilized by a companion unit to recognize a person in an environment. For example, the companion unit could disclose the name of such a person to the user if the user is having memory issues. As another example, if the companion unit flies to the door when someone is at the door of the building where the user is located, the companion unit may use the facial recognition operability to identify the person at the door by capturing an image of a person through a window. The unit may then relay the identify of the person at the door to the user so the user can decide whether or not to answer the door.

In some embodiments of the present invention a microphone array may be used that is operable to locate the source of a voice. Upon locating the voice the unit may aim one or more of the cameras towards to source of the voice. In this manner one or more images, or video feed, of the speaker may be captured by the one or more cameras. The detection of the voice source and/or the aiming of the one or more cameras may occur when the companion unit is stationary, such as when it is positioned on a base or upon a surface, or when the unit is in-flight.

As shown in FIG. 2, a chassis 26 may be incorporated within the interior of the companion unit. Moreover, other elements of the companion unit may further be recessed wholly or partially into the interior of the companion unit, such as the one or more speakers 20, the one or more batteries 24, the one or more microphones 22, the one or more cameras 24, and the circuit board.

Furthermore, the one or more batteries 28 may be recessed in the interior of the companion unit. The one or more batteries are operable to power the companion unit. The batteries may be rechargeable, and integration of the companion unit with the docking station base of the companion system may instigate recharging of one or more of the batteries. The one or more of the batteries of the companion unit may be charged inductively, or by direct contact with a charging unit.

As shown in FIG. 3, the one or more navigational sensors may assist with the flight of the companion unit. As an example, two proximity sensors 30a and 30b may be integrated with the unit. The proximity sensors may sense objects or surfaces proximate to the unit, such as by emitting an electromagnetic field of, or a beam of, electromagnetic radiation (e.g., infrared), and detecting changes in the field or return signal. The navigational sensors may further be of the type to engage stereo video ranging, optical flow analysis, light detection and ranging (LIDAR), infra-red (I.R.) sensor arrays, laser ranging, ultrasonic ranging and/or other methods or combinations of methods. A skilled reader will recognize that other types of proximity sensors can be integrated in the companion unit to recognize objects or surfaces proximate to the unit. Such recognition may permit the companion unit to choose a flight path that is free from objects and surfaces that may otherwise block the flight path of the companion unit. This increases the safety and effectiveness of the companion unit and system as it avoids crashes of the unit that may lead to incapacitation or malfunction of the companion unit.

The companion unit may further incorporate other types of navigational sensors, such as ranging and obstacle avoidance sensors 32 that detect obstacles in the flight path of the unit, or other navigational sensors. For example, the navigational sensors may further allow the user to determine the distance between the companion unit and a user to ensure that the companion unit does not collide with the user. For example, if the companion unit is commanded to follow the user, the navigational sensors may be used to ensure that the companion unit flies or hovers in a manner that maintains a distance between the user and the companion unit. This distance should be kept constant or virtually constant while the companion unit follows the user, even as the user starts, continues (possibly at a varied speed) and stops movement.

In some embodiments of the present invention the companion unit may further incorporate one or more altitude sensors 34, as shown in FIG. 4. The altitude sensor is operable to detect the altitude of the flight of the companion unit, and to detect the distance between the base of the companion unit and a surface below the companion unit, such as a floor 36. The altitude sensor may be utilized to detect surfaces and objects below the companion unit that may hinder or otherwise affect the flight of the companion unit.

The companion unit may be operable to perform obstacle avoidance and altitude hold. It may further incorporate navigation modules that allow for other flight or hover operability. The navigation modules may be incorporated in the companion unit, the companion system or the artificial intelligence module (AI Module), or connected to any of these, whereby instructions may be provided to the companion unit.

As discussed herein, embodiments of the present invention may operate with beacons that are located in an environment, such as Bluetooth™ beacons, or other types of beacons, to achieve navigation.

The present invention may generate a map of an area proximate to a user, such as a building or an outdoor area. The location of the companion unit may be shown on the map, and the movement of the companion unit within the space may be tracked on the map. The map may indicate obstacles in the space, and the map may be utilized by the companion unit and system to cause the companion unit to avoid obstacles.

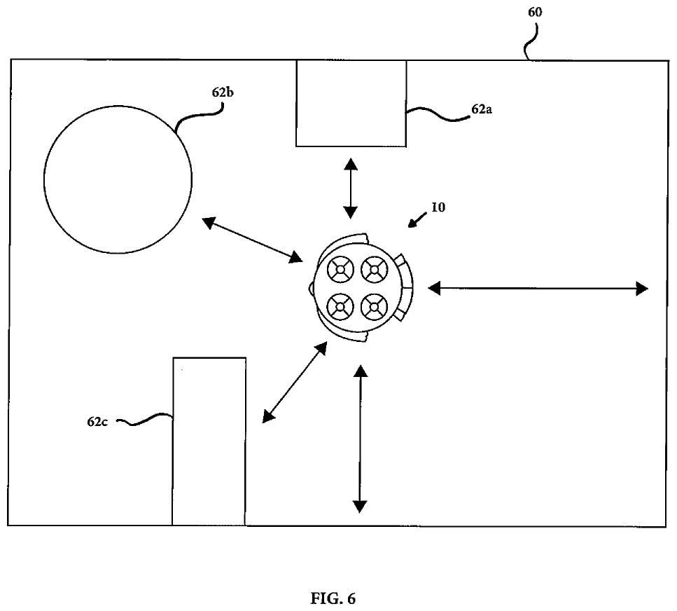

For example, as shown in FIG. 6, the navigational sensors of the companion unit 10 may be used to detect obstacles 62a, 62b, 62c within a room 60 or other area that are proximate to the unit. In particular, the navigational sensors collect the distances and direction of reflections of the fields or beams emitted from the sensors. The companion unit may utilize this information to construct a map of an area, to locate the unit within the mapped area, and to track the location of the unit as it moves within the area. Beacons may also be utilized in the mapping of an area.

In one embodiment of the present invention the companion unit may incorporate a processor operable to generate the map. The processor of the companion unit may further be operable to update the map, and update the position of the companion unit upon the map in real-time or virtually real-time.

In another embodiment of the present invention, a processor may be integrated remotely from the companion unit, such as in the base unit, in a computer device that is connected to the companion unit and can transfer information to and from the unit through such connection, and/or in a device located remotely from the user that is connected to the companion unit and can transfer information to and from the unit through such connection. For example, the connection may be a WiFi connection or some other type of connection operable to transmit information to and from the companion unit and system.

The processor(s) of the companion unit and system may control multiple operations of the companion unit and system described herein, including the generation of a map of an environment proximate to a user. The processor may generate the map and make the map available to the unit. Information that can be processed to identify the location of the unit within the mapped environment may be transferred from the navigation system to the processor, and once the location of the companion unit within the mapped environment is identified this information can be transferred to the navigation system. In this manner, the map can be generated and the location of the companion unit within the map can be identified, tracked and updated. The map tracking the location of the companion unit may further be displayed to the user or to another person via a computer device. In this manner if the user sends the companion unit to a different room in the building than the room where the user is located, the user can watch the movement of the companion device once it is out of the user's room by viewing the map on the computer device. A map may be stored in memory and re-used if at a later point in time the companion unit enters the same environment that was previously mapped.

In yet another embodiment of the present invention, the operations of generating the map, identifying the location of the companion unit within the map, and updating the location of the companion unit within the mapped environment, may be divided between two or more processors. One processor may be located in the companion unit, and one or more other processors may be located remotely, such as in one or more base units, and/or in other computing devices. The operation of each of the processors may be determined in accordance with how information can be efficiently transferred and processed for the operation of the companion unit and system. For example, efficient transfer and processing may be achieved by a configuration that is operable to achieve speed in the transfer and processing operations of the companion unit and system. Other functions of the companion unit and system may also be divided between multiple processors.

The memory of the companion unit and system, wherein data, information, and software code of the companion unit and system is stored, may be located within the companion unit in some embodiments of the present invention. In other embodiments of the present invention the memory may be remotely located from the companion unit, such as within the base unit, or within one or more remote servers. In still other embodiments of the present invention, the memory may be divided between multiple locations. For example, memory may be located within the companion unit, within one or more base units, within one or more mobile devices (e.g., a cell phone, a smart phone, a tablet, a computer device, a WiFi enabled device, etc.), and/or at one or more remote servers.

When a previously generated map is re-used the companion unit may need to continue to use its navigational sensors to detect proximate obstacles. This may be necessary as objects in the mapped space could have been moved since the map was originally generated.

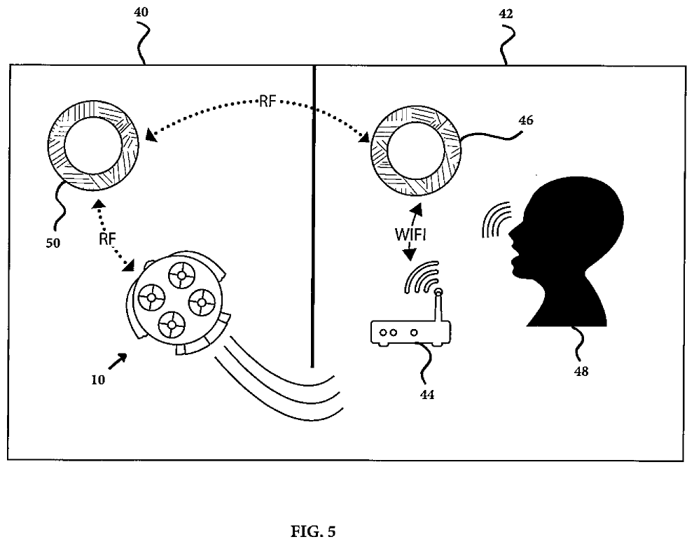

The companion system may incorporate several elements. As shown in FIG. 5, the companion system may incorporate multiple base units, including a docking station base 46, and one or more satellite bases 50. The system may further incorporate at least one wireless local networking (WiFi) unit 44. The companion unit may be able to recharge its batteries on one or more of the bases, including the docking station base and any of the satellite bases. Such one or more bases operable to recharge the companion unit will incorporate a recharging capability for the battery of the companion unit by direct contact or inductively.

The companion unit may further receive upgrades and/or modifications to any software or other data stored within the companion unit while connected with any of the bases. As an example, such upgrades or modifications may include improvements to the capability or function of any of the elements incorporated in the companion unit, such as expanded audio capabilities that would provide greater speaker, microphone and processing capability, or any other modifications or upgrades to elements of the companion unit.

As discussed herein, the configuration of the processor(s) and storage unit(s) of embodiments of the present invention may vary, and upgrades and modifications to software or other data may be achieved in accordance with the configuration of the companion unit and system. For example, a software upgrade may be transferred to the processor or storage unit where such software is located (e.g., within the companion unit, or remotely from the companion unit). The transfer may direct, or may involve transfer through multiple elements of the companion unit and system.

The one more bases, of any type, may be configured to rotate. This allows for the orientation of the companion unit to be modified while the companion unit is at rest in connection with the base. The rotation may further permit the companion unit to be positioned so that it can conduct surveillance of the user, such as video or image surveillance, while the companion unit is at rest (i.e., not flying). For example, the base may rotate to direct one of the cameras of the companion unit to places of interest in the environment. The base may be motorized so that its rotation is controlled by a motor element. The orientation of the base may occur in accordance with control by the user or another person, such as through use of a handheld controller, or the orientation of the base may be controlled through the processing of information by the companion unit and system. For example, one or more cameras of the companion unit may be operable to identify the location of the user or other persons in the environment, and the orientation of the base may be controlled in relation to the location of the user or one of such other persons.

The base may have an appropriately sculpted form which reduces air turbulence affecting the companion unit during take-off and landing of the companion unit therefrom. The base may incorporate one or more sensors that guide the companion unit during landing upon the base.

The companion system may integrate one or more satellite bases and these can be located in multiple rooms within a building or within multiple locations within an area. The companion unit can land upon all bases, and the batteries of the unit can be recharged upon any base that has recharging functions. The base may be operable to charge the companion unit inductively.

One or more of the bases may have speakers incorporated therein or otherwise connected thereto that are operable to deliver sound, and/or one or more microphones incorporated therein or otherwise connected thereto that are operable to capture and possibly record sound.

As shown in FIG. 5, the companion system elements may be positioned so as to be spread over multiple rooms 40, 42 within a building. For example, the docking station base may be in a different room than a satellite base. A radio frequency (RF) will travel between the docking station base and the satellite base, whereby information can be transferred between the docking station base and the satellite base. There may also be a RF connection between the companion unit and whichever satellite base or docking station base that it is closest to, whereby the companion unit and base each transmit and receive information. For example, when the companion unit is closer to the satellite base, it will be receiving and transmitting RF between itself and the satellite base.

The WiFi unit may provide the docking station base with Internet connectivity, whereby data may be transferred to and from servers and/or computing devices via the Internet by the docking station base. In the example configuration of an embodiment of the present invention shown in FIG. 5, interne connectivity provided by the WiFi unit is transferred to the docking station base, and information transferred via the WiFi unit is further transferred by RF to the satellite base, and from the satellite base to the companion unit. Therefore, it may be possible for an Internet podcast to be received by the docking station base via the WiFi unit, and transferred to the satellite base whereby it is transferred to the companion unit. Once received by the companion unit, the companion unit can cause the podcast to be audible in the environment of the companion unit through the one or more speakers in the companion unit.

As another example of use of the companion system, the user 48 may provide voice commands that are received by the docking station base (or whatever base is closest in proximity to the user), and the commands may be transferred via RF signals to the companion unit. The system will process the commands and act in accordance with the commands. For example, if the voice command is for a particular podcast to be played, the command may be processed by the system, such that the podcast is obtained via the Internet and broadcast through the speakers of the companion unit.

In embodiments of the present invention, voice interaction may occur mainly when the companion unit is located upon a base. This will prevent the noise of the motor of the companion unit from interfering with the voice transmission and detection. However, embodiments of the present invention can be configured to engage in voice interaction when the companion unit is located upon the base, as well as when the companion unit is in-flight.

The companion system may be controlled through any of user voice interaction, automated central command modules, a computer device (e.g., smart phone, laptop, computer, tablet, a smart television, a WiFi enabled appliance, or other computer device). The companion system may further operate with a voice command product that functions to undertake particular tasks upon voice commands, such as playing music, providing navigational directions, or providing other information such as news, sports scores, weather reports, or other information obtained directly from a storage source in the voice command product (e.g., Apple™ Siri, Amazon Echo^{TM}, or other voice command products), or otherwise via the Internet. The companion system may generate voice commands that are directed to the voice command product to generate a response to such voice command by the voice command product. The user may also deliver voice commands to the voice command product, and the companion unit may capture the information output by the voice command product, such as a weather report, news, or other information through one of the microphones of the companion unit. The companion unit may store such information collected from the voice command product and/or use such information in the processing of the companion unit and system.

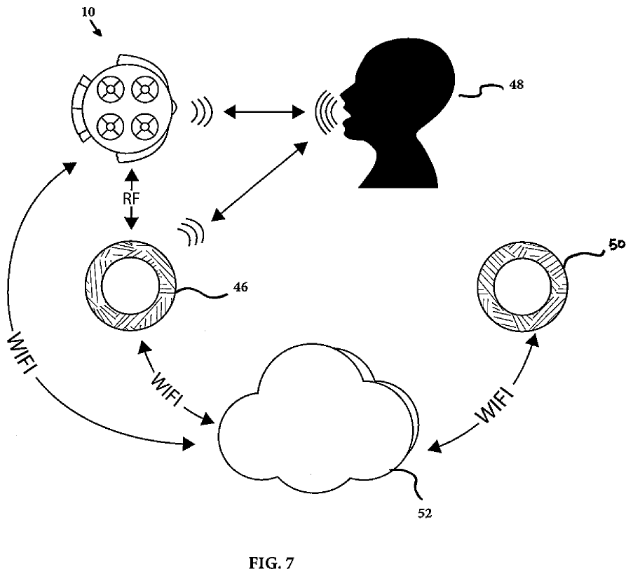

An example of control by user voice interaction is shown in FIG. 7. The user 48 provides voice commands and those commands are collected by the base and/or by the companion unit. The companion unit in particular collects the voice command though the microphone which transfers the collected and possible recorded sound to its circuit board. The voice command may be transferred to a processor for processing to determine the command to be acted upon or the information to be responded to that was provided through the voice command. A voice command can also be stored in the one or more of any storage units integrated with the companion unit and system.

As shown in FIG. 7, a WiFi connection to the Internet may provide access to cloud storage 52 whereby a voice assistant or artificial intelligence elements may be accessed. The elements of the companion system, the companion unit, satellite base and docking station base, may each be individually connected to the cloud storage. RF connections may also exist between the elements, such as a RF connection between the companion unit and the base that it is closest thereto (e.g., as shown in FIG. 7 to be the docking station base). The voice commands may be processed by the voice assistant and/or artificial intelligence element, and instructions as to the activity that the companion unit is to undertake in response to the voice command are sent back to the companion unit directly, or indirectly, such as via a base, via a WiFi connection to the voice assistant and/or artificial intelligence element (AI Module).

In an embodiment of the present invention, one or more of the voice assistants may be operable to apply speech recognition and speech synthesis processing. The results of this recognition and processing can be provided to the AI Module, and in turn by the AI Module to provide instructions to the companion unit. In this manner the response to voice commands provided to the AI Module directly, or to the companion unit and/or the companion system, can produce the required activity and response by the companion unit.

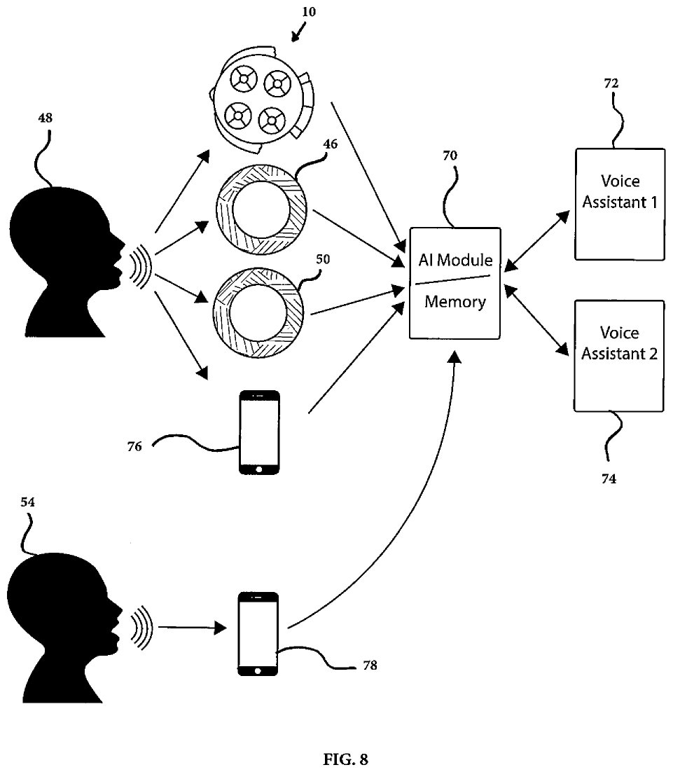

Due to the interconnectivity of the elements of the companion system, as shown in FIG. 8, the user 48, may provide voice commands to multiple elements of the companion system, either simultaneously or individually, including to the satellite base 50 and/or to the docking station base 46. The user can also provide voice commands to the companion unit. Another element that the user could use to provide voice commands is a cell phone 76, or some other computer device (i.e., a smart phone, a tablet, a computer, a laptop, a smart television, a WiFi enabled appliance, or any other computer device) that is connected to an artificial intelligence module 70 (the “AI Module”).

The AI Module functions to cause continuous improvement and learning by the companion unit system with respect to the interactions between users and the companion unit. The AI Module incorporates software code and is linked to a database of statistical pointers and weights pertaining to past conversational interactions. Such statistical pointers and weights are accessible by the AI Module to diminish aspects of interactions of the companion unit with the users that detract from the flow of such interaction. For example such detractors that may be diminished include repetition of questioning, facts or other areas for which the user has provided the companion unit with a prior correction to address an assumption or confused meaning, and other detractors from conversational flow and/or accuracy of topic and language between users and the companion unit. The statistical pointers and weights can be utilized to iron-out such detractors and cause the companion unit not to incorporate such detractors in future interactions with users. This can result in improvements to the voice natural language interface of the companion unit, whereby the companion unit speaks to and converses with users.

In addition to identifying detractors, the AI Module may be operable to identify topics or areas relating to which the companion unit can spontaneously initiate conversation, as well as topics and/or areas where some deep learning can be applied to a trained model of the AI Module to better predict a user's interests or preferences. In embodiments of the present invention, the deep learning and development of trained models with voice interaction can be performed on a cloud server as a background task.

In embodiments of the present invention, trained models, networks, and statistical databases can be aggregated for one or more deployed companion units to produce aggregated data. The aggregated data can incorporate learning achieved by multiple companion units, such as corrections to meanings of words used in conversations. In this manner, a companion unit can apply to its interactions with users learning that was achieved by another companion unit. This can cause the improvement and learning of a companion unit to be beyond that achieved by said individual companion unit based upon interactions of that companion unit with one or more users. The aggregated data can thereby be used to improve the voice interface of the companion unit.

The companion unit is operable to achieve capture of a user's information. This captured information can be processed by the AI Module to cause learning by the AI Module that improves the interaction between the companion unit and the user. The AI Module acts as a controller of the interaction of the companion unit with the user. The AI Module can decide what mix of capabilities, vocal responses, video capture, security, entertainment, and communication features to employ, and what flying mission, and destination to select for a companion unit. The AI Module can suggest new flying missions for a companion unit, and adapt to changes in environment.

In some embodiments of the present invention, the AI Module can generate and create new flying missions in relation to specific circumstances existing for interaction between a companion unit and a user. The AI Module is operable to interpret the information required to define the desired behaviour of the companion unit. The behavior may be desired by a user, or be desirable in light of other factors. The AI Module is operable to communicate the desired behavior to the flight control module and thereby to effect a flight path and flying behaviours of the companion unit.

The AI Module incorporates software operable to receive and process voice commands, or commands provided in other formats (i.e., text, images, computer code, etc.). The AI Module is further operable to transmit the results of the processing that are output as commands to the companion system and/or the companion unit. The AI Module may incorporate or be connected (through a wired or wireless connection) to a memory and/or data storage such as one or more drives or servers. The remote controller may provide voice commands to the AI Module. The AI Module will process the received commands.

For example, the AI Module may be connected to one or more voice assistants modules 72, 74. The voice assistant modules may be operable to receive voice commands, to process the voice commands, and to thereby create output that is a conversion of the voice commands into instructions the AI Module can receive and use to perform certain functions. The AI Module may transfer such instructions to the companion unit to cause the companion unit to perform one or more functions.

As an example, the user's voice command could be “Follow me” and the AI Module, using one or more of the voice assistant modules, may cause instructions to be provided to the companion unit to begin to fly behind the user as the user moves through an area. As another example, if the user's voice command is to play music, the AI Module, using one or more of the voice assistant modules, may cause instructions to be provided to the companion unit whereby music stored in the companion unit, or music stored in the cell phone 76 or in another local computer device, or music accessible via the Internet, to be played through the speakers of the companion unit. As discussed herein, instructions may be provided directly to the companion unit, or the instructions may be provided indirectly to the companion unit, such as via one or more of the companion system elements with delivery to the companion unit being the final delivery in a series of deliveries.

The AI Module may further create instructions based upon triggers either stored in the AI Module data storage, or provided by the companion system to the AI Module. Such triggers may cause the AI Module to send an instruction to the companion unit whereby the unit is triggered to pose a question or a comment to the user, such as a question or a comment relating to medications the user is required to take, or other questions or comments. The trigger may also be for the companion unit to undertake an activity, such as recording video of the user, or capturing images of the user.

The AI Module may further be triggered to generate instructions to the companion unit whereby the companion unit is directed to fly through the house for a security sweep of the house. For example, the companion unit may generate video that is transferred to the AI Module. The AI Module may process the video to confirm that doors and windows are closed in the building where the user is located (the building wherein the companion unit is flying). The AI Module may further generate instructions whereby the companion unit interacts with the user, and such interaction is initiated by the companion unit. Such interactions may be verbal interactions, such as the companion unit asking the user if the user needs any music played, or any other activity to be undertaken by the companion unit. The interactions may be created to ward off loneliness if the user is alone. The companion unit may be triggered to start a conversation with the user by a variety of triggers, such as the length of time when there is a lack of sounds in an environment, a response to particular information received by the companion unit, a timed trigger, or any other trigger.

In an embodiment of the present invention, the companion system may connect via WiFi to WiFi enabled appliances located proximate to the companion unit. The companion system may act as a conduit for communication with a WiFi enabled appliance. Therefore, the user may transfer commands to a WiFi enabled appliance via the companion system. In this manner the present invention may be integrated with the internet of things.

In some embodiments of the present invention the companion system may be controlled from a remote location, by a caregiver, health professional, or family member of the user, or by some other person who the user authorizes to access and control the companion system (a remote user). As an aspect of controlling the companion system, the person will be able to control the companion unit. As discussed herein, there are multiple ways to control the companion unit, and therefore the companion unit may be controlled by a user and one or more other people simultaneously, such that the companion unit will collect information and will undertake certain activities in response to commands or control by the user and multiple other people.

A person who controls the companion system who is located remotely from the companion system, a remote controller 54 (remote user), may use a cell phone 78, or another computer device (i.e., a smart phone, a tablet, a computer, a laptop, a smart television, a WiFi enabled appliance, or any other computer device) to access the AI Module 70. The connection from the remote controller's cell phone to the AI Module may be via an Internet connection, or via a cell connection, or in any other manner. The remote controller may provide voice commands to the AI Module via the cell phone 78. The AI Module may process the voice commands, such as with the assistance of one or more voice assistants, and may transmit the results of the processing as output commands to the companion system and/or the companion unit. For example, the remote controller may provide a voice command whereby the companion unit provides video of the user to the remote controller's cell phone. As another example, the remote controller may provide instructions whereby the companion unit performs a security check of the building where the user is located, or plays particular audio or video entertainment for a user.

As yet another example, the remote controller may ask for confirmation that the user has taken their medications. Such a request may involve the companion unit being triggered previously in the day to capture video of the user taking their medications and sending this video to the AI Module. The AI Module can either send the video to the remote controller, or it may process the video to confirm that the user took their medications through image recognition software and provide the remote controller with a response to their query.

As still another example, the remote controller may provide voice commands whereby the companion unit can permit the remote controller to have a conversation with the user via the remote controller's cell phone, the speaker of the companion unit, and the microphone of the companion unit that can capture the user's voice and this can be transmitted via the companion system to the AI Module and via the AI Module to the remote controller.

A skilled reader will recognize that there are a variety of voice commands that can be provided by the remote controller, and that the AI Module, the companion system, and the companion unit can be programmed to respond to these voice commands in a variety of ways, including the AI Module sending a response to the remote controller based upon data collected relating to the user, or the companion unit can be caused to perform an activity in relation to the remote controller's voice command (e.g., fly a mission, etc.).

In embodiments of the present invention video may be captured by the companion unit and transferred to a computer device. The video may be captured by one or more of the one or more cameras 24 integrated in the companion unit 10. The video may be transferred from the companion unit to the base unit that the companion unit is closest to (whether that be the docking station base 46, or one of the satellite bases) via the RF connection. The base may transfer the video to a computer device 80 (i.e. a computer, a laptop, a tablet, a smart phone, a cell phone, a smart television, a WiFi enabled appliance, or another computer device). The computer device may be connected to the Internet via a WiFi unit 44. The computer device may transfer the video to one or more other computer devices, such as a cell phone 82 (located in the area of the companion unit), a WiFi enabled television 84, a remote cell phone 86, or any other device operable to receive video. The transfer of the video may be via the Internet, directly via WiFi, or through any other transmission connection between the computer device transferring the video and the computer device receiving the video. The video can then be displayed on the computer device that receives the video if it incorporates a screen or via a screen connected to said computer device.

As described herein, video captured by the companion unit can be used to perform a safety sweep of the home. If the video is transferred to a screen where it is viewable by a person (e.g., a user, a remote controller, or some other person), that person may undertake to view the video and thereby confirm that all doors and windows are shut in a building, that locks are being utilized in a building, that no unexpected person is in the building, and any other security related information that can be gleaned from viewing the video. In a similar manner, the video captured by the companion unit as it is flying may allow a viewer of the video to locate an item in a building or area (such as an outdoor area) that was previously misplaced, or the location of a pet within a building or area.

Video can be used for other purposes as well, such as monitoring a user, to determine if they are showing any signs of distress, if they have fallen, if they are having a medical emergency, etc. Video can be used by health professionals for diagnostic purposes, as the companion system and unit may be used to provide video of a user to a health professional and/or to allow the health professional to speak with the user while viewing the user. A skilled reader will recognize that there are other uses of video that can be made in an application of the companion system.

Through the companion system elements and the artificial intelligence module, the companion unit can act as a companion to a user. The companion system and/or companion unit can be controlled from outside of the location of the user, such as outside the home of the user, an outdoor area where the user is located (e.g., a backyard or another outside area), or a combination of outdoor and indoor areas proximate to the user.

When the companion unit and system are controlled remotely, a person located remotely from the user can interact with the user and/or monitor the user. As an example, the companion system could be used by a caregiver to obtain some respite time, while still allowing the caregiver to be able to monitor the safety and health of the user. This could be of great importance to the health of the caregiver, as the physical and mental burden of caring for a person who is in ill-health can be significant, and respite from such caregiving activities can be critical to the health of the caregiver.

The companion unit and system may be operable for a variety of uses. For example, it can be used to can deliver entertainment, as well as purposes of security, advice, information, communication with family, caregiver or health professionals, medical monitoring performance, confirmation of medical compliance, as well as general surveillance. The operability of the companion unit and system to engage with persons located remotely from the user further provides remote family and caregivers with peace of mind as they are able to monitor and/or interact with the user. The companion unit and system can supplement a traditional caregiver and allow a caregiver to be remotely located from the user. As the companion unit and system can act as an interface for communication with the user, the caregiver is not out of visual or oral communication with the user, even if the caregiver is distantly located from the user.

The companion unit and system further are operable to combat isolation and loneliness of a user, in particular a user who is ill, housebound, or aged. The companion unit provides companionship to the user, as described herein.

The companion unit may be operable to navigate by itself as it is moving within a building or an area. In some instances the companion unit may be controlled by voice commands of the user or another person to navigate within a space. However, it could also be human controlled through the connection of traditional UAV navigation controls to the companion system or directly to the companion unit, or through a handheld controller.

The settings for control of the companion unit and system may be determined on a case-by-case base, in accordance with the user preferences, the needs of the caregiver or family members, and the requirements proscribed by any healthcare provider working with the user. There may also be standard settings programmed into the companion unit, such as user directed settings, family caregiver directed settings, medical caregiver directed settings, and/or other person settings.

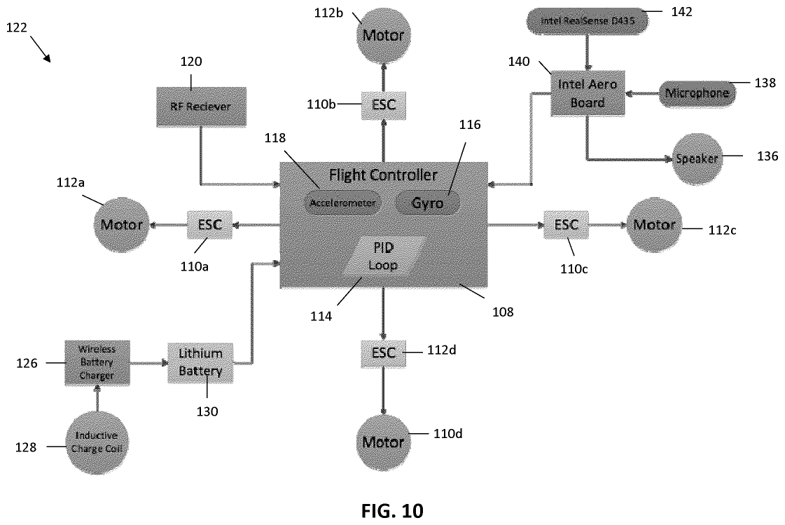

Embodiments of the present invention may be operated and controlled via a control system 122, as shown in FIG. 10. In such a system a flight controller 108 is operable to control the flight of the companion unit and system. The flight controller incorporates several elements operable to control the stability, acceleration, and flight path of the companion unit.

The flight controller and the elements incorporated therein, operate in a coordinated manner to control the flight, including hovering, of the companion unit. The flight controller receives information and other data from the companion unit relating to the speed, direction, altitude, intended destination, and other information pertaining to flight and movement of the companion unit while the companion unit is in-flight or hovering.

The flight controller, and the elements incorporated therein, utilize the information received from the companion unit to determine if adjustments should be made be to the flight of the companion unit (e.g., its direction, speed, altitude, destination, and other information relating to its flight and movement) to cause the flight of the companion unit to be stable and consistent along the flight path that is determined for the companion unit to cause the companion unit to reach its destination. The flight path of the companion unit may be determined by the flight plan guidance unit of a function system of the present invention, or another element of the companion unit and system of this present invention. The flight path may be generated based upon the intended destination of the companion unit and any known obstructions (e.g., trees, buildings, walls, windows, etc.) between the companion unit and its intended destination. The flight controller may be integrated in the function system of the present invention, or within the companion unit and system generally. The flight controller may further operate with sensors and other components that detect objects that could affect the flight of the companion unit along a flight path, as discussed herein.

The flight controller is operable to receive data for multiple sensors and external devices, and to process such data to determine the optimum commands to provide to the companion unit in order for it fly or hover, as required for its function to assist the user. The flight controller incorporates a processor that receives data and information, and processes this data and information through the application of computer software programmed to perform calculations and other operations. The sensors that provide data to the flight controller, to be processed by the flight controller processor, may include one or more of the following: a magnetometer; a barometer; an airspeed sensor; a distance sensor; an optic flow sensor (whereby the position of the companion unit is maintained in-flight); and any other sensors utilized to assist the flight of the companion unit to be stable and consistent with the flight path of the companion unit.

The flight controller incorporates an accelerometer 118 operable to measure in-flight acceleration of the companion unit and stabilize the flight of the companion unit. The flight controller further incorporates a gyroscope 116, operable to facilitate smooth flight and hovering by the companion unit. The smooth flight and hovering operability of the companion unit is critical to the image capture features of the present invention, as it ensures that images captured are not shaky or blurry.

The companion unit can be subjected to a number of forces coming from different directions when in-flight. For example, the companion unit's yaw, pitch, and roll can be affected by such forces. As a result of such forces the flight of the companion unit can be hard to control. The gyroscope operates to virtually instantly detect changes in the in-flight position of the companion unit and compensate for such changes in position. The gyroscope operates to facilitate a flight path for the companion unit that is virtually unaffected by the forces. This can be achieved by the gyroscope performing adjustments to the companion unit's position many times in quick succession, such as hundreds of times every second, that cause the companion unit to remain along a flight path or to hover in place.

The flight controller 108 of embodiments of the present invention may incorporate a proportional-integral-derivative controller (PID Loop) 114. The PID Loop is operable as a control loop feedback mechanism that continuously calculates an error value as the difference between a desired setpoint and a measured process variable. The PID Loop applies a correction based on proportional, integral and derivative terms. The PID Loop is operable to stabilize the attitude (e.g., altitude, etc.) of the companion unit in-flight when the companion unit is influenced by forces, by correcting the propulsion system of the companion unit. The PID Loop will automatically apply accurate and responsive corrections to control the flight of the companion unit, including corrections to the speed of the companion unit, via control of the power output of the motor of the companion unit.

RF signals transmitted from the companion unit may be received by an RF receiver 120 operable to transmit such RF signals to the flight controller 108. The flight controller may utilize such RF signals as information (e.g., commands) applied to initiate certain functions of the flight controller and the elements incorporated therein.

The flight controller may be powered by a battery or direct connection to a power outlet. If the flight controller is powered by a battery, the battery may be a rechargeable battery 130, such as a lithium battery or another type of rechargeable battery. If the battery is a rechargeable battery, an inductive charge coil 128 may be connected to a wireless battery charger 126, and the inductive charge coil and wireless battery charger collectively are operable to inductively recharge the rechargeable battery. The recharable battery powers the flight controller. Recharging of the rechargeable battery may occur when the power of the rechargeable battery reaches a particular level (which may be sensed by the wireless battery charger), on a regular basis (e.g., bi-daily, daily, overnight, or some other regular basis whereby the rechargeable battery is sufficiently charged to power use of the companion unit as required by the user thereof), or at other intermittent, or random, points in time.

The flight controller 108 may be connected to an areo board 140, operable to connect to one or more sensors and peripheral devices (e.g., microphones, speakers, etc.). In some embodiments of the present invention, the areo board may be an Intel™ areo compute board, or some other areo board. The areo board may incorporate or be connected to one or more sensors, for example, such as a magnetometer and an altimeter. Information collected by the sensors that is transmitted to the areo board can be utilized and processed by a processor incorporated in the areo board. The sensors incorporated in the areo board, or otherwise connected thereto, are operable to perform sensing operations. For example, the altimeter will sense the altitude of the companion unit and provide information relating thereto that is utilized by the processor of the areo board. As another example, the areo board may incorporate a sensor that is a camera operable to support HD colour and infra-red depth sensing features.

The areo board may be connected to one or more external sensors and/or peripheral devices. The areo board may be connected to a tracking sensor unit 142. In some embodiments of the present invention, the tracking sensor unit may be an Intel™ realsense device, or another tracking sensor unit. The tracking sensor unit may be operable to facilitate depth perception functions relative to the in-flight location of the companion unit. The depth perception functions enable the companion unit to “see” and understand the world, and such information can be utilized by the AI Module of the present invention to assist with the learning of the companion unit and system. The tracking sensor unit may incorporate vision processors, depth and tracking modules and depth cameras. In some embodiments of the present invention, the tracking sensor unit may be incorporated in the companion unit. The tracking sensor unit is operable to send information collected by the depth sensors (e.g., information relating to the depth of field between the companion unit and other objects), and other information collected by the other sensors of the tracking sensor unit, to the areo board.

The areo board may receive information transmitted from one or more peripheral devices, including a microphone 138. A user may provide commands to the companion unit via the microphone, or a remote user may relay a message or other vocalized information to be delivered to a user of the companion device via a microphone. Such commands or other vocalized information may be transmitted from the microphone to the areo board, for example, such as in the form of sound waves or a digital equivalent thereof.

The areo board processor may be operable to process the microphone information transmitted to the areo board into speaker output. The areo board is operable to transmit the speaker output to a speaker, whereby the vocalized information provided via the microphone is audible via the speakers. For example, if a remote user provides vocalized information to be delivered to a user, the areo board may send such information to a speaker such that it is delivered to the user as audio output of the speaker. The areo board may further process information it receives from sensors and/or peripheral devices via operation of the areo board processor. The areo board processor generates information to be transferred to a speaker to be delivered to the user as audio output of the speaker. Such information may be warning information, notification information, or other information generated based on the information collected by the sensors. In embodiments of the present invention, the speakers may be one or more speakers, and one or more of such one or more speakers may be incorporated in the companion unit.

The processor of the areo board may process the information it receives from all sensors and peripheral devices connected to the areo board. The processed information may be transmitted from the areo board to the flight controller. In some embodiments of the present invention, the areo board may transmit information it receives from sensors and/or peripheral devices to the flight controller without processing such information.

The flight controller, and the elements incorporated therein, utilize the information received from the areo board to stabilize, direct and otherwise control the flight and hovering of the companion unit. The flight controller 108 is operable to transmit information to an electronic speed control (ESC) 110a, 110b, 110c, 110d that may be positioned in any direction from the flight controller (as indicated in FIG. 10 wherein the

ESC connected to a motor is shown to be positioned in four different possible directions from the flight controller).

The ESC is operable to receive information relating to adjustments to the speed of the companion unit and to transmit such information to the motor 112a, 112b, 112c, 112d of the companion unit, whereby the speed of the companion unit is adjusted in accordance with the information transmitted by the ESC. The ESC thereby regulates the speed of the motor of the companion unit. The ESC is also operable to cause the motor to function to perform braking, or to reverse the motor of the companion unit (whereby the direction of the flight of the companion unit may be reversed).

In this manner the flight controller generates and receives information that is utilized to control the flight of the companion unit. The flight controller may be incorporated in a function system of the present invention.

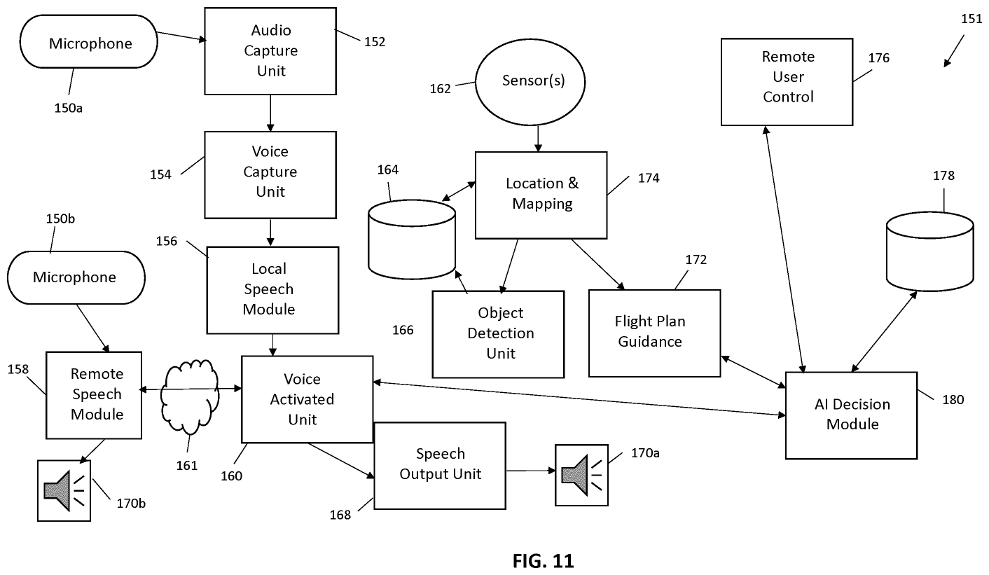

Embodiments of the present invention may incorporate a function system 151, such as is shown in FIG. 11. The function system is operable to receive commands from the user, remote users, other people, the internet of things, and/or the companion unit and/or other elements of the companion system. The function system is further operable to generate flight plans and otherwise generate a response to the command(s) it receives. The function system incorporates multiple elements, including computer-implemented elements that are operable to receive and transmit information, and to process information. Such computer-implemented elements may be connected to external devices that either: transmit information to one or more computer-implemented elements; or to which at least one of the computer-implemented elements transfer information. Such information received by the external device(s) may produce an activity or function by such external devices,

External devices may include any one of, all of, or any combination of, the following: microphones, cameras, speakers, sensors, computer devices, and any other external device operable to collecting information or transmit information.

An external device that is a microphone 150 may be utilized by a user, whereby a user transmits audio information via the microphone. For example, the user may speak into, or in the vicinity of, the microphone and the audio information (e.g., voice, sounds, music, etc.) of the user may be detected by the microphone. Such audio information (e.g., speech by a voice) may incorporate a command for the companion unit.

The microphone 150a may be incorporated in a companion unit or otherwise incorporated in the system of the present invention generally. The microphone is connected to an audio capture unit 152. The audio information is transmitted from the microphone to the audio capture unit. In some embodiments of the present invention, there may be one or more microphones connected to the audio capture unit, each microphone being operable to detect audio information and to transmit such audio information to the audio capture unit.

The audio capture unit is a computer-implemented element that incorporates a processor. The processor of the audio capture unit is operable to capture the audio information transmitted via a microphone as vocalized information. For example, the audio capture unit may process the vocalized information to remove or diminish ambient noise, or to process the vocalized information in other manner. The audio capture unit 152 is connected to a voice capture unit 154. The audio capture unit is operable to transmit the processed vocalized information to the voice capture unit.

The voice capture unit is a computer-implemented element incorporating a processor. The voice capture unit is operable to process the processed vocalized information transmitted from the audio capture unit, to distinguish the voice of the user from the vocalized information and to thereby generate speech information. The voice capture unit 154 is connected to the local speech module 156. The speech information may be transmitted from the voice capture unit to the local speech module.

The local speech module is a computer-implemented element incorporating a processor. The processor of the local speech module is operable to generate commands from the speech information. The local speech module 156 is connected to a voice activated unit 160 that is a computer-implemented element. The commands are transmitted by the local speech module to the voice activated unit. A command may be a command for an activity to be undertaken, a query request for a query to be undertaken, or a comment that results in a response (e.g., in the form of conversation), or other voice input that elicits some type of response from the voice activated unit. The voice activated unit may respond to the commands.

If the response of the voice activated unit to a command includes providing an audio response to the user, the voice activation unit may send speech output information to a speech output unit 168 that is connected to the voice activated unit 160. The speech output information is processed by a processor of the computer-implemented speech output unit and transmitted as audio output to one or more speakers 170a that are connected to the speech output unit. The audio output is provided to the user via the one or more speakers as voice or other audio (e.g., music, sounds, or other audio).

In embodiments of the present invention, a remote speech module 158 is a computer-implemented element that may be connected to the voice activated unit 160. The remote speech module may receive audio input via a microphone 150b that is used by a remote user. For example, the microphone 150b may be incorporated in a smartphone or other computer device utilized by the remote user. The remote user may convey audio input via the microphone that is to be delivered to: the user who is located in proximity to a companion unit; a companion unit; the flight controller; or the system generally. The audio input is processed by the remote speech module, whereby the audio input is delivered to the intended recipient to produce the results intended by the remote user.

The remote user may engage in a conversation with the user, and in such a circumstance the audio input of the remote user may be transmitted by the voice activated unit to the speech output unit and therefrom to the one or more speakers, as described herein. In such a circumstance it may further be possible for the user to provide responses to the remote user via the microphone in proximity to the user, and for such responses to be: processed as described herein; and recognized by the voice activated unit as a command that indicates that the voice activated unit should transmit such audio output to the remote speech module. The remote speech module is a computer-implemented element operable to provide such audio output to a remote user via one or more speakers 170b that are proximate to the remote user. For example, the one or more speakers may be incorporated in the smart phone or other computer device that the remote user utilizes to connect to the remote speech module. In some embodiments of the present invention, the remote speech module may be connected to the voice activated unit via the Internet 161 or a cloud service.

The voice activated unit may be a voice assistant as described herein. For example, the voice activated unit may be an Amazon™ voice service, a Google Home™ service, or any other type of voice assistant. The voice activated unit is operable to receive and process voice input that is a command, and to undertake particular functions in relation to such voice input.

As discussed herein, the voice assistants may be operable to apply speech recognition and speech synthesis processing. In accordance with the function system, these processes may be undertaken by any one, or a combination, of the computer-implemented elements between the microphone 150a and the voice activated unit, namely: the audio capture unit, the voice capture unit, and the local speech module if the microphone that was used was connected to the audio capture unit; or the remote speech module if the microphone that was used was connected to the remote speech module.

In some embodiments of the present invention, a voice assistant may comprise an audio capture unit 152, voice capture unit 154, local speech module 156, voice activated unit 160 and speech output unit 168.

The voice activated unit 160 is connected to the AI decision module 180. The AI decision module is operable to transmit information to the voice activated unit. The voice activated unit may be operable in accordance with such information to generate particular speech to be delivered to a user via the speech output unit and the speaker. In this manner, the speech delivered to a user may be generated in accordance with learning of the AI Module that facilitates interaction between a user and the companion unit, as discussed herein.

The results of the processing of the voice activated unit, or any other information transmitted to the voice activated unit, can be provided to the AI decision module (that is a computer-implemented element and is an element of the AI Module). As described herein, the AI decision module may be operable to provide instructions to the companion unit via the AI Module. In this manner the response to voice commands provided to the AI decision module directly, or to the companion unit and/or other elements of the companion unit system, can produce the required activity and response by the companion unit. The AI decision module is connected to one or more servers 178, and information provided to, or generated by, the AI decision module can be transmitted to the one or more servers for storage. Such information can also be accessed from the one or more servers by the AI decision module.

One or more sensors 162 may be connected to embodiments of the present invention. In some embodiments of the present invention, one or more sensors may be incorporated in the companion device. One or more sensors may further be incorporated in other elements of the present invention or otherwise connected to the function system of the present invention. The information collected by each of the sensors will be in accordance with the function of such sensor. For example, an altitude sensor will collect information relating to the altitude of the companion device, and other sensors will collect other information in accordance with the operation of each type of sensor. A variety of types of sensors can be incorporated in embodiments of the present invention, as discussed herein.

The sensor(s) 162 are connected to a location and mapping unit 174 that is a computer-implemented element. The information collected by the sensor(s) is transmitted to the location and mapping unit. The location and mapping unit is operable to construct or update a map of an unknown environment while simultaneously keeping track of the companion unit's location within said environment. In embodiments of the present invention, the location and mapping unit may be a simultaneous localization and mapping unit (SLAM) that is tailored to the companion unit and system of the present invention.

The location and mapping unit incorporates a processor operable to process the sensor information and to generate flight information. A map may be generated by the location and mapping unit that includes at least the area proximate to the companion unit (e.g, the room in a house where the companion unit is located, the ground floor of a house where the companion unit is located, a field outdoors where the companion unit is located, or some other area proximate to the companion unit). The map may be transferred to the one or more location and mapping servers 164 connected to the location and mapping unit 174. The location and mapping unit is operable to access maps and other information stored in the one or more location and mapping servers. In this manner, maps generated previously can be accessed and such previously generated maps can be updated by the location and mapping unit.

The location and mapping unit 174 transmits the flight information to a flight plan guidance unit 172, and information is further transmitted to the flight plan guidance unit from the AI decision module. The flight plan guidance unit is a computer-implemented element and it incorporates a processor operable to process the information it receives from the location and mapping unit (information that includes readings from the one or more sensors) and from the AI decision module (information that includes the required destination(s) of the companion unit based on commands from the user, remote users, or commands that are generated by the system of the present invention based on an event or some other criterion).

The flight plan guidance unit generates a flight plan for the companion unit, in accordance with the required destination(s), that will assist the user. For example, the flight plan may be generated upon a command from a user for the companion unit to check that all of the windows in a building are closed. The flight path may be generated whereby the companion unit will fly to each of the windows in the building, and hover near the windows to use video or other technologies to identify the windows as closed or open. The flight plan guidance unit incorporates the flight controller, whereby the companion unit is controlled to fly along the flight path in a stable manner, as described herein.

The location and mapping unit 174 is connected to an object detection unit 166, and the location and mapping unit may transmit information to the object detection unit. The object detection unit is a computer-implemented element and it may incorporate a processor operable to process the information received from the location and mapping unit. Such processing may detect objects in an area for which the location and mapping unit has generated a map. In embodiments of the present invention, the object detection unit may be a rCNN™ Object Detection unit, or some other object detection unit. The object detection unit modifies a deep neural network that was originally trained for image classification using millions of annotated images to be utilized for the purpose of object detection. In general terms, the object detection unit operates to identify regions of interest (ROIs) in a map, review most of the identified ROIs, and classify and label each reviewed ROI. The processor applies ROI pooling whereby an ROI is projected onto a convolutional feature map. The process detects object locations at different scales and aspect ratios. The results of the object detection processing, that identifies objects within a mapped space, can be saved to the one or more location and mapping servers 164 connected to the object detection unit.

In some embodiments of the present invention, a remote user control unit 176 may be operable to receive commands from a remote user, whereby a remote user can control the companion unit via the function system. The remote user control unit is a computer-implemented element. The remote user commands may be provided through a website connected to the function system, or via the remote user's computing device connected to the function system. The commands may be provided via audio, text or other input into said computing device connected to the function system. Remote user commands may also be provided via the internet of things.

Remote user commands may be commands to control the flight of the companion unit, whether to control the flight of the companion unit upon a flight path in-flight, to change the flight path of a companion unit in-flight, or to identify a destination and path thereto for a flight path for a companion unit. The remote user commands are transmitted to the AI decision module connected to the remote user control unit. The AI decision module is operable to transfer such commands as information to the flight plan guidance unit, in the manner described herein.

The AI Module further can be utilized to develop a personality for the companion unit. Various settings for types of personalities (e.g., talkative, introverted, extroverted, etc.) can be incorporated in the AI Module and exhibited by the companion unit. The type of personality the companion unit exhibits can be chosen as a setting, and such a setting can be changed in the same manner as other settings are changed relating to the operability of the AI Module.

Whatever personality is chosen, the AI Module is operable so that the personality of the companion unit is adaptive, and the companion unit will learn the user's preferences and operate in accordance with such preferences. As an example, user preferences that can be learned by the companion unit and system include the times of the user's favourite television or radio programs, the friends who the user likes to call, preferred music the user likes to listen to, the types of humour and comments that elicit a positive response from the user, as well as other types of preferences. Examples of how the companion unit, through interaction with the AI Module, can learn to act in response to these preferences include that the companion unit may interact with a WiFi enabled television to tune into one or more of the user's favourite television programs. As another example, the companion unit may play music that is preferred by the user. As yet another example, the companion unit may learn to interact with the user in a manner that includes discourse that integrates the user's preferred type of humour. The personality and capabilities of the companion unit can be expanded by central control, or by the user, through the function of the AI Module.

The personality of the companion unit may be adaptable to the user's speech patterns, speech content, and preferences. Different personalities and voices for the companion unit are selectable by the user at the outset, but the personality of the companion unit and system develops in complexity through use thereof and learning of the user's preferences. By interrogating the user, the personality module gathers information about preferences relating to the user, and it can download additional capabilities to meet those preferences. Tasks and capabilities of the companion unit and system may be expandable by downloading applications.

It will be appreciated by those skilled in the art that other variations of the embodiments described herein may also be practiced without departing from the scope of the invention. Embodiments of the present invention may be utilized by and/or in relation to users who are children, elderly persons, housebound persons, persons who are ill, or any other person. Embodiments of the present invention may further be developed for long-range outdoor use. Such embodiments may incorporate GPS, be configured to have an extended power charge for longer operation of the flight controller and companion unit between battery charging, have longer range communications capabilities, and have other features relating to outdoor use.

We claim:

1. A companion system operable to interact with a user, comprising:
(i) a companion unit that is an unmanned aerial vehicle incorporating one or more navigational sensors, one or more cameras, one or more speakers and one or more microphones;
(ii) one or more bases wirelessly connected to the companion unit; and
(iii) an artificial intelligence module (AI Module) connected to the companion system operable to process commands and provide instructions to the companion system to be performed by the companion unit.

2. The companion system of claim 1, further comprising the one or more navigational sensors that include one or more proximity sensors operable to recognize obstacles in the flight path of the companion unit.

3. The companion system of claim 1, further comprising one or more of the following being connectable via an Internet connection: the companion unit; and at least one of the one or more bases.

4. The companion system of claim 3, further comprising one or more mobile devices remotely located from the companion unit being connectable to one or more of the following via the Internet: the companion unit; and at least one of the one or more bases, said one or more mobile devices being operable by one or more remote users to communicate with the companion unit and to the user via the companion unit.

5. The companion system of claim 4, further comprising the one or more remote users being one or more of the following: a care giver; a health care provider; a family member; or a friend.

6. The companion system of claim 1, further comprising one or more of the following being connectable to one or more computer devices: the companion unit; and at least one of the one or more bases.

7. The companion system of claim 6, further comprising the one or more computer devices being one or more of the following: a cell phone, a smart phone, a laptop, a tablet, a smart television, and a WiFi enabled appliance.

8. The companion system of claim 1, further comprising a flight controller connected to the companion unit, whereby in-flight speed and direction of a companion unit may be controlled.

9. The companion system of claim 1, further comprising the one or more sensors connected to a location and mapping module operable to generate a map local to the location of the companion unit.

10. The companion system of claim 9, further comprising a flight plan guidance unit operable to receive information from the AI Module and the map, and to process such information and the map to generate a flight path for the companion unit.

11. The companion system of claim 1, further comprising a voice assistant operable to receive voice commands provided via the microphone of the companion unit, and to process said voice commands to cause the companion unit to respond by flying a mission or generating a voice response provided to the user via the speakers of the companion unit.

12. A method of one or more operating users operating a companion unit, comprising the following steps of:
(i) one of the one or more operating users providing a command to the companion unit;
(ii) the companion unit transmitting the command to an artificial intelligence module (AI Module) and the AI Module processing the command to generate a response that is transmitted to the companion unit; and
(iii) the companion unit operating in accordance with the response.

13. The method of claim 12, comprising the further step of one of the one or more operating users, being a local user in proximity to the companion unit, providing the command as a voice command to a microphone of the companion unit.

14. The method of claim 12, comprising the further steps of:
(i) one of the one or more operating users, being a remote user remotely located from the companion unit, providing the command as a voice command to a microphone in a computing device of said remote user;
(ii) the computing device transmitting the voice command to one of the following: a microphone of the companion unit, and the microphone transmitting the voice command to the AI Module; or the AI Module; and
(iii) the AI Module processing the voice command as the command to generate the response.

15. The method of claim 12, comprising the further steps of:
(i) one of the one or more operating users, being a remote user remotely located from the companion unit, providing the command as an input command by inputting the input command into a computing device of said remote user;
(ii) the computing device transmitting the input command to the AI Module; and
(iii) the AI Module processing the input command as the command to generate the response.

16. The method of claim 12, comprising the further step of the AI Module transmitting information to a flight controller, and said flight controller controlling a motor of the companion unit to control the flight of the companion unit that is the response to the command.

17. The method of claim 16, comprising the further steps of:
(i) one or more sensors transmitting sensor information to the flight controller; and
(ii) the flight controller processing such sensor information to control the flight of the companion unit.

18. The method of claim 12, comprising the further steps of:
(i) one or more sensors transmitting sensor information to a location and mapping unit and the location and mapping unit processing the sensor information to generate a local map of an area proximate to the companion unit;
(ii) the location and mapping unit transmitting the map to a flight guidance unit;
(iii) the AI Module transmitting command information to the flight plan guidance unit;
(iv) the flight plan guidance unit processing the command information and map to generate a flight path within the map for the flight of the companion unit that is the response to the command; and
(v) the companion unit flying along the flight path to execute the response.

19. The method of claim 12, comprising the further steps of the AI Module transmitting information to one or more databases; and the AI Module accessing information from the one or more databases, said information being utilized by the AI Module in processing the command and generating the response.

20. The method of claim 12, comprising the further steps of:
(i) one or more cameras incorporated in the companion unit generating video of an area proximate to the companion unit and transferring said video to one or more computer devices for viewing by the one or more operating users;
(ii) the one or more operating users communicating with each other via microphones and speakers incorporated in the companion unit; and
(iii) the companion unit engaging in conversation with at least one of the one or more operating users.
